(19) United States
(12) Patent Application Publication
(10) Pub. No.: US 2018/0125423 A1
(43) Pub. Date: May 10, 2018

(54) SYSTEM AND METHOD FOR ACTIVITY MONITORING EYEWEAR AND HEAD APPAREL

(71) Applicant: Lumo BodyTech Inc, Mountain View, CA (US)

(72) Inventors: Andrew Robert Chang, Sunnyvale, CA (US); Chung-Che Charles Wang, Palo Alto, CA (US); Ray Franklin Cowan, Mountain View, CA (US)

(21) Appl. No.: 15/805,416

(22) Filed: Nov. 7, 2017

Related U.S. Application Data
(60) Provisional application No. 62/418,436, filed on Nov. 7, 2016.

(51) Int. Cl. A61B 5/00; A61B 5/11

(52) U.S. Cl. CPC A61B 5/6803; A61B 5/1114; A61B 5/746; A61B 5/486; A61B 5/7282; A61B 5/1118

(57) ABSTRACT

A system and method for activity monitoring eyewear and head apparel can include collecting kinematic data from an activity monitoring system that is coupled to a user head region; generating a set of activity signals that comprises at least one head orientation signal that is at least partially generated from the kinematic data head orientation signal from the kinematic data; monitoring the set of activity signals for a response condition; and triggering an action response upon detection of the response condition.

### CROSS-REFERENCE TO RELATED APPLICATIONS

This Application claims the benefit of U.S. Provisional Application No. 62/418,436, filed on 7 Nov. 2016, which is incorporated in its entirety by this reference.

### TECHNICAL FIELD

This invention relates generally to the field of activity monitoring wearables, and more specifically to a new and useful system and method for activity monitoring eyewear and head apparel.

### BACKGROUND

A variety of computer devices such as smart phones and wearables have been introduced and achieved wide public adoption in recent years. Newer devices such as smart glasses and virtual reality headsets are beginning to be more commonly used by the public. These recent trends in computing devices have led to device interactions that can be damaging to the body of the user. For example, many people suffer from neck injuries from looking down at their phones for extended periods of time. The introduction of augmented and virtual reality devices that rely on head movement for computer interactions will expose similar or even more dangerous risks. Thus, there is a need in the activity monitoring wearables field to create a new and useful system and method activity monitoring eyewear and head apparel. This invention provides such a new and useful system and method.

### BRIEF DESCRIPTION OF THE FIGURES

FIG. 1 is a schematic representation of a system of a preferred embodiment;

FIG. 2 is a schematic representation of a system variation including a secondary monitoring system;

FIG. 3 is a schematic representation of an exemplary head orientation mapping;

FIG. 4 is a schematic representation of use with a secondary computing device like a smart phone;

FIG. 5 is a schematic representation of augmenting a secondary computing device;

FIG. 6 is a schematic representation of activity classification;

FIG. 7 is a graph comparison of exemplary kinematic data from two activities;

FIGS. 8A and 8B are schematic representations of eyewear in different resting states;

FIGS. 9 and 10 are graph representations of exemplary kinematic data from different item interactions;

FIG. 11 is a graph representation of exemplary kinematic data with different processing modules working in combination;

FIG. 12 is a flowchart representation of a method of a preferred embodiment;

FIG. 13 is an exemplary two dimensional orientation map;

FIG. 14 is an exemplary three dimensional orientation map;

FIG. 15 is an exemplary two dimensional orientation map showing metrics resulting from different types of head orientation; and

FIG. 16 is an exemplary schematic representation of head orientations and resulting orientation map representations.

### DESCRIPTION OF THE EMBODIMENTS

The following description of the embodiments of the invention is not intended to limit the invention to these embodiments but rather to enable a person skilled in the art to make and use this invention.

### 1. Overview

A system and method for activity monitoring eyewear and head apparel of preferred embodiments function to generate biomechanical signals from kinematic data that can be used in directing interactions of the user. The resulting interactions of the system and method may be applied in a variety of ways including but not limited to user ergonomics feedback, activity analytics for a user, eyewear/headphone/headwear usage and design augmentation, device usage ergonomics feedback, device usage safety feedback, augmenting of secondary computing functionality (e.g., interacting with a phone, augmented reality headset, virtual reality headset, etc.), and/or other suitable applications.

The system and method are particularly applicable to use-cases where the kinematics (e.g., orientation and movement) of a user's head and/or neck are measured and used to at least partially determine resulting interactions. Through the system and method, connected eyewear, headphones, or other forms of headwear can be powered by a biomechanical signal sensing device platform that can include hardware, software algorithms, applications, and services used to drive various interactions by tracking the motion of the head and detecting the micro-movements of a head worn device.

The system and method may be applied to a variety of head-based form factors such as eyewear, headphones, hearing aids, ear buds, earrings, hats, helmets, and/or other head-worn items.

Eyewear may include vision corrective eyewear, sunglasses, safety goggles, connected/enhanced glasses, a virtual reality (VR)/augmented reality (AR) headset, a glasses frame, or other forms of eyewear. In one connected/enhanced glasses variation, eyewear can be basic glasses or frames with subtly integrated electronics used for providing the kinematic sensing and processing described herein. In another connected/enhanced glasses variation, the eyewear can be smart glasses which may include the system and method in addition to audio capabilities, tactile feedback, a display, touch controls or other forms of user input, and other elements.

Headphones may include normal audio headphones with integrated sensing technology, but can additionally include smart headphones with additional capabilities, hearing aids and/or other devices with head-worn audio devices. For example, the system and method could be integrated into headphones where audio and/or tactile feedback may be used in place of visual, audio, or tactile feedback of a smart glasses implementation.

Herein, eyewear is primarily used as the example form factor, but the system and method could alternatively make use of any suitable form factor.

Additional intelligence of the system and method embedded into the glasses or other head-worn item allows for a more robust understanding of user activity and how glasses or the item are used in the real world. Such data can be used to build new personalized experiences for users. In particular for glasses, but also applicable to other head-worn items, the system and method can provide more accurate data to designers to help better design items that better fit different facial profiles and under different activity conditions.

Exemplary uses of the system and method can include: mitigating neck pain; strengthening the neck and/or enhancing flexibility; detecting posture and providing feedback; encouraging users to be more active; tracking daily activities; determining when a new pair of glasses are needed; designing better glasses for different people and/or activities; personalizing the design of glasses for different facial structures; recommending different glasses; altering interactions when using phones, computers, the TV, augmented/virtual reality; reinforcing safe driving practices while in a car; and/or other applications.

As one potential benefit, the system and method may enable an activity monitoring and a biometric feedback system that is naturally or even transparently integrated with a head worn product.

Another set of benefits can include the variety ways in which the system and method can be applied.

A potential benefit of one set of applications of the system and method may include the generation of ergonomic/posture feedback. The system and method may be used in guiding the correction of posture. The system and method may also be used in generating targeted exercises that can be incrementally or selectively delivered in spaced sessions. For example, a variety of different micro stretch/exercise sessions can be delivered over the course of a day to enhance the strength and flexibility of a user and/or mitigate damaging posture or sustained load.

A potential benefit of another set of applications of the system and method may include augmentation of device usage based on detected head-related motion or activity. A secondary device that is communicatively coupled to an activity monitoring system may be a focus of interaction for a user, and the system and method may alter those interactions to promote better posture and/or avoid chronic problems. In some variations, this may be done through notifications/communications to the other device. In other variations, this may be achieved through active manipulation of a secondary device. For example, the system and method may be used to reposition windows or automatically scroll a scrollview to reposition the focus of a user interface to alter the direction of a user's gaze.

A potential benefit of another set of applications of the system and method may include collection of product usage analytics. Physical usage of devices like glasses and headphones can be detected and tracked. This may be used to empower designers and product makers to gain a better understanding of their products. Such product usage analytics can additionally be used to generate recommendations based on how previous users used a product.

### 2. System

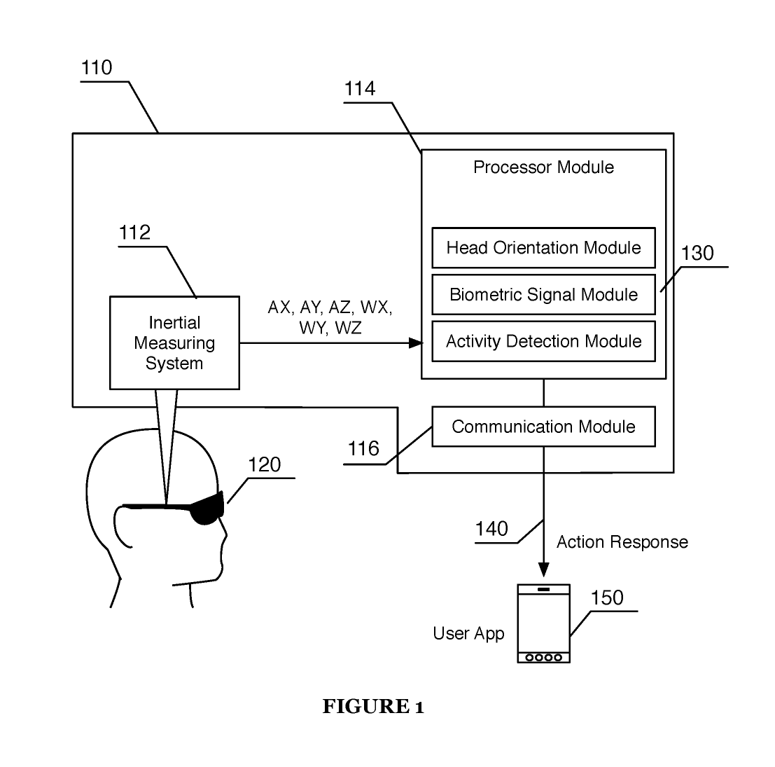

As shown in FIG. 1, a system for activity monitoring eyewear and head apparel of a preferred embodiment can include an activity monitoring system 110 integrated into a head-wearable item 120, a set of biomechanical processing modules 130, and at least one feedback interface 140. In one implementation, the system can include an application 150 communicatively coupled to that of the activity monitoring system 110. The activity monitoring system 110 and the application 150 can operate cooperatively in configured processing of collected kinematic data and generation of resulting interactions.

An activity monitoring system 110 of a preferred embodiment functions to collect kinematic data that is then transformed to one or more activity signals such as biomechanical signals. In particular head orientation is a preferred biomechanical signal. The biomechanical signals sometimes in combination with other inputs can be used to trigger or direct interactions of the system. The activity monitoring system 110 can include an inertial measurement unit 112, a processor 114, and optionally a communication module 116. The activity monitoring system 110 can additionally include any suitable components to support computational operation such as a processor, data storage, RAM, an EEPROM, user input elements (e.g., buttons, switches, capacitive sensors, touch screens, and the like), user output elements (e.g., status indicator lights, graphical display, speaker, audio jack, vibrational motor, and the like), communication components (e.g., Bluetooth LE, Zigbee, NFC, Wi-Fi, cellular data, and the like), and/or other suitable components.

The activity monitoring system 110 may serve as a standalone device where operation is fully contained within the activity monitoring system 110 and the head-wearable item 120. The activity monitoring system 110 may additionally or alternatively communicate with at least one secondary system such as an application operating on a computing device; a remote activity data platform (e.g., a cloud-hosted platform); a secondary device (e.g., a mobile phone, a smart watch, computer, TV, augmented/virtual reality system, etc.); or any suitable external system.

The inertial measurement unit 112 functions to measure multiple kinematic properties of an activity. In particular, the inertial measurement unit 112 generates kinematic data reflecting the movements of a user's head. An inertial measurement unit 112 can include at least one accelerometer, gyroscope, magnetometer, and/or other suitable inertial sensor. The inertial measurement unit preferably includes a set of sensors aligned for detection of kinematic properties along three perpendicular axes. In one preferred variation, the inertial measurement unit 112 is a 9-axis motion-tracking device that includes a 3-axis gyroscope, a 3-axis accelerometer, and a 3-axis magnetometer. The sensor device can additionally include an integrated processor that provides sensor fusion. Sensor fusion can combine kinematic data from the various sensors to reduce uncertainty. In this application, it may be used to estimate orientation with respect to gravity and may be used in separating forces or sensed dynamics for data from a sensor. The on-device sensor fusion may provide other suitable sensor conveniences. Alternatively, multiple distinct sensors can be combined to provide a set of kinematic measurements. In some variations, the system may include multiple activity monitoring systems and/or inertial measurement units 112 that are positioned at different portions of the body. Some portion of these additional sensing locations may be used in detecting particular biomechanical signals.

An inertial measurement unit 112 and/or the activity monitoring system 110 can additionally include other sensors such as an altimeter, GPS, or any suitable sensor. Additionally, the system can include a communication channel to one or more computing devices with one or more sensors. For example, an inertial measurement unit can include a Bluetooth communication channel to a smart phone, and the smart phone can track and retrieve data on geolocation, distance covered, elevation changes, land speed, topographical incline at current location, and/or other data.

The processor 114 functions to transform sensor data generated by the inertial measurement unit 112. The processor 114 can include a calibration module and a set of biomechanical signal monitors possibly including head-calibrated orientation sensing, a step segmenter, activity classification, and/or other biomechanical signals. The processing can take place on the activity monitoring system no or be wirelessly transmitted to a smartphone, computer, web server, and/or other computing system that processes the biomechanical signals.

The processor 114 used in applying signal processing on the kinematic data can be integrated with the activity monitoring system no. For example, a wearable device with a battery, a communication module, and some form of user control can generate the biomechanical signals on a single device like glasses or a headphone. The processor 114 may alternatively be application logic operable on a secondary device such as a smart phone. In this variation, the processor 114 can be integrated with the user application. In yet another variation, the processor 114 can be a remote processor accessible over the network. Remote processing may enable large datasets to be more readily leveraged when analyzing kinematic data.

The communication module 116 functions to relay data between the head-worn activity monitoring system 110 and at least one other system. The communication module 116 may use Bluetooth, WiFi, cellular data, and/or any suitable medium of communication. For example, the communication module 116 can be a Bluetooth chip with RF antenna built into the device. As discussed, the system may be a standalone device where there is no communication module 116.

The system can additionally include one or more feedback elements, which function to provide a medium for delivering real-time feedback to the user. A feedback element can include a haptic feedback element (e.g., a vibrational motor), audio speakers, a display, or other mechanisms for delivering feedback. Other user interface elements for input and/or output can additionally be incorporated into the device such as audio output elements, buttons, touch sensors, and the like. Feedback can be delivered through a system integrated with the head-wearable item 120 or a secondary computing device such as a smart phone, smart watch, a computer, and/or another suitable computing device.

A head-wearable item 120 functions to physically couple an activity monitoring system no to the head region of a user when in use. The activity monitoring system no in one variation may be removably attached to a head-wearable item. In this removable variation, the activity monitoring system 110 can include a body with an attachment mechanism so that it can be substantially stably connected to the head-wearable item 120. Alternatively, the head-wearable item 120 may include an attachment mechanism to hold or otherwise fix an activity monitoring system no. For example, a head-wearable item 120 may include a defined cavity configured to hold an activity monitoring system no. For example, a defined concave cavity with side indents can be configured for an activity monitoring system 110 to selectively snap in and out of the cavity.

In another variation, the activity monitoring system no may be integrated into the design of the head-wearable item 120. For example, glasses may have the activity monitoring system 110 integrated into frames of the glasses. In another example, headphones may have the activity monitoring system no integrated into the earpiece and/or band of the headphones.

The head-wearable item 120 may come in a variety of form factors. The head-wearable item 120 can be any type of device that is usually worn on a human head. This includes but not limited to eyewear including various types of eyeglasses, goggles, sunglasses, or VR/AR headset. The head-wearable item 120 may alternatively include headwear such as headphones, hearing aid, earbud, headband, hairclip, hat, helmet, earrings, a hood, or anything that can be worn on the head securely.

In both variations, the physical coupling of the activity monitoring system may be substantially fixed relative to the general region and orientation of the head. For example, the activity monitoring system 110 may attach to a region around the ears or between the eyes for glasses. In another example, headphones may have a fixed location for the activity monitoring system about the ear region or at some point across the band.

The activity monitoring system 110 may act independently, but may alternatively operate in connection with one or more other devices or systems such as a connected application, a secondary monitoring device 160, and/or a secondary computing device 170.

The application 150 functions as one potential outlet of the biomechanical signal output. The application 150 is preferably used in combination with the activity monitoring system no to facilitate interactions with the user and/or coordinate processing and synchronization of data. The user application 150 can be any suitable type of user interface component. An application 150 is preferably user accessible on a personal computing device as a native application 150 or as an internet application 150. Preferably, the user application 150 is a graphical user interface operable on a user computing device. The user computing device can be a smart phone, a desktop computer, a TV based computing device, a wearable computing device (e.g., a watch, glasses, etc.), or any suitable computing device. The user application 150 can alternatively be a website accessed through a client browsing device. Alternatively, the biomechanical signals may be accessed synchronously or asynchronously through an application programming interface (API).

The application 150 can allow the user to sync data from the activity monitoring system no, receive user feedback, configure settings, and view the data from the device. The application 150 can also process the kinematic data, processed data, or biomechanical signal data from the device. The application 150 can additionally facilitate communication with a webserver that can sync data, send firmware updates, or additional context such as social comparisons with other users to create a more compelling user experience. Haptic feedback elements, text-based notification, audio feedback and/or other forms of user feedback may additionally be performed or controlled by the application 150.

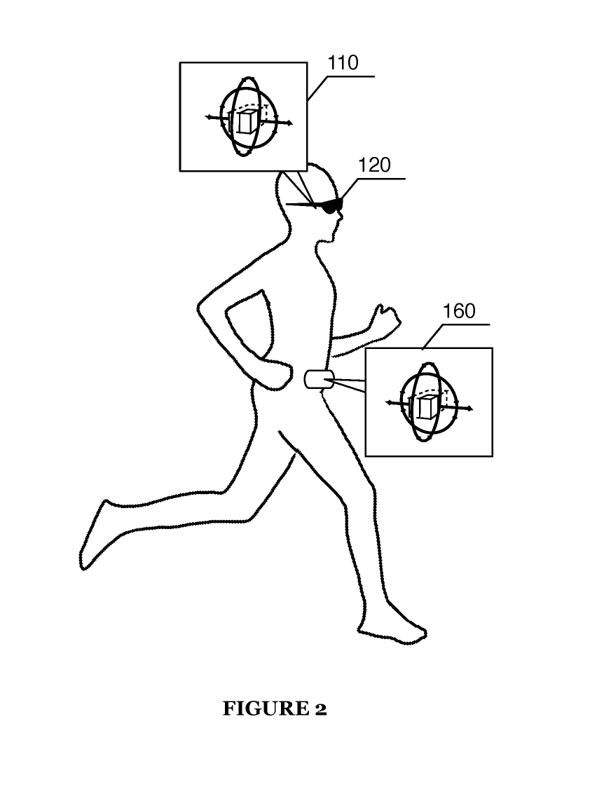

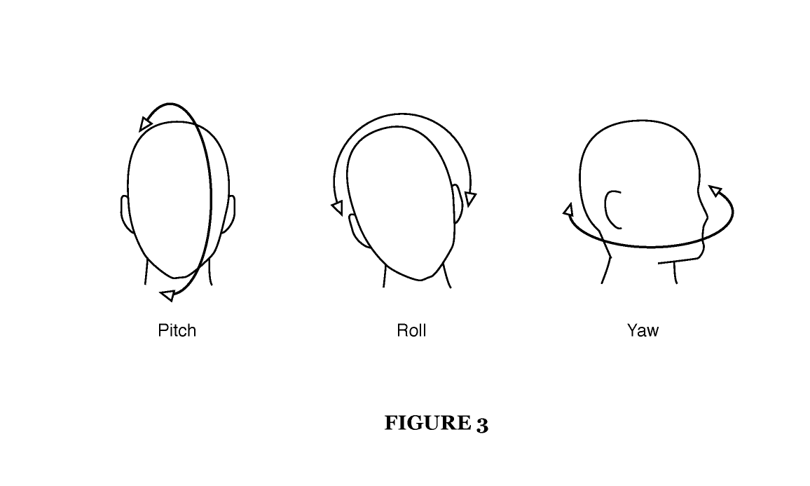

The secondary monitoring device 160 functions as a device to establish a frame of reference for inferring head-based biomechanics as shown in FIG. 2. In some scenarios detection of some aspects of head movements or positions may be enhanced by establishing movement and position of the body. The secondary monitoring device 160 preferably collects kinematic data or other forms of data to determine motion and/or orientation of the lower body (e.g., below the neck). This may be applied to differentiating head rotation and movement from body rotation and movement. For example, a secondary monitoring device 160 may detect yaw rotation (as characterized by the pitch, roll, yaw orientations shown in FIG. 3) of 30° and the activity monitoring system 110 (i.e., the primary monitoring device) may measure 50° yaw rotation. The system can then infer a head yaw rotation of 20°.

The secondary monitoring device 160 is preferably another kinematic data sensing element including some portion of an inertial measurement unit. In one variation, the secondary monitoring device 160 may be substantially similar to the activity monitoring system no described above. In another variation, the secondary monitoring device 160 may provide a more limited scope of sensed data. For example, the secondary monitoring device 160 could include a magnetometer so that body direction can be determined.

The secondary monitoring device 160 is preferably physically coupled to the torso region of the user such as the chest, stomach, upper or lower back, pelvis, etc. The torso region may provide less variation from arm or leg movement when compared to the head region. The secondary monitoring device 160 could alternatively be coupled to the arm, hand, leg, or foot. Accommodation of limb movement is preferably accounted for in these variations. In one variation, body orientation may be periodically calibrated and measured during particular windows that are based on easier to measure conditions. Additionally, the kinematic signals measured at the activity monitoring system 110 may provide a point of reference for the secondary monitoring device 160 as well.

In some variations, the secondary monitoring device 160 can be a dedicated device in communication (via a wired or wireless connection) with the device activity monitoring system no wherein one of the two devices may act as the master/controlling device. In a related variation, the secondary monitoring device 160 could also be a personal computing device wherein an application on the personal computing device acts as the secondary monitoring device 160. In some implementations, a smart phone or a smart watch could provide the as the base monitoring device of the lower body. For example, a smart phone may provide a suitable reference point when in a user's pocket or held in the user's hand during use.

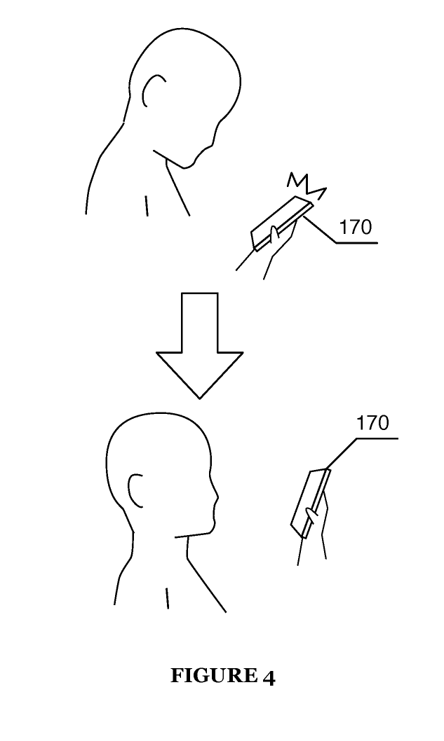

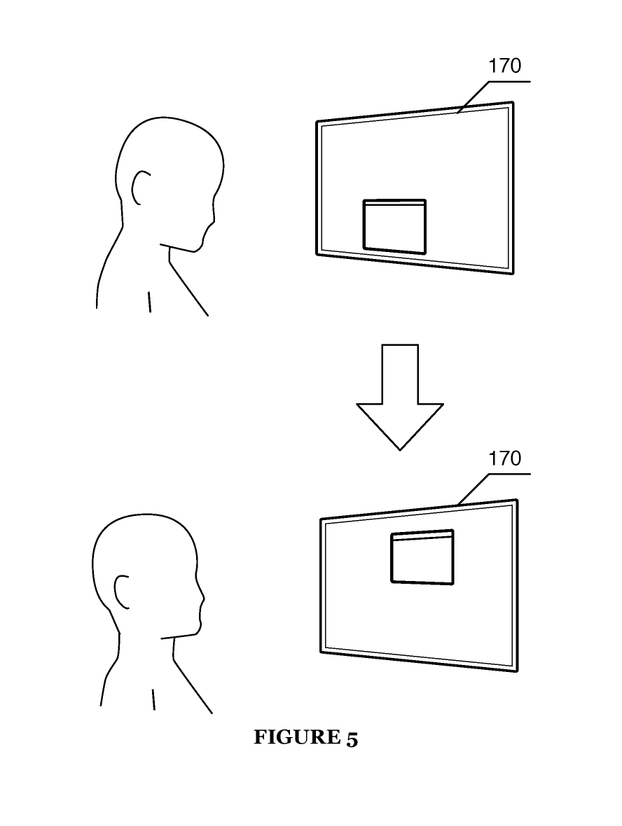

A secondary computing device 170 functions to be a device that may serve as a subject for monitoring user activity or being the output of feedback. The use of electronic devices over extended periods of time are a large contributor to the recent trends in back and neck problems. Some variations of the system may have integrated access to such a device to understand biomechanics of the head region during device usage. The secondary computing device 170 can be a smart phone, a computer screen, a virtual display space for an AR or VR device, or any suitable computing device. Computing devices that require directed attention, thereby directing or biasing the positioning of the head, may be of particular interest for integration as a secondary computing device 170 of the system as shown in FIGS. 4 and 5. The secondary computing device 170 may offer some alternative purpose or use such as use a smart phone or providing AR or VR experiences.

The secondary computing device 170 preferably includes a system integration enabling usage monitoring and/or optionally application control, which can be used in augmenting user experiences with the device. Integration with the secondary computing device 170 may be an application, a background service, an operating system level feature, hardware integration, or any suitable form of integration. User activity may indicate use of the device (e.g., mouse input, keyboard input, touch input), user of a particular application, configuration of the device or application (e.g., positioning of the windows or different views), orientation and motion of the device, and/or other aspects pertaining to the user of the device. In some variations, the secondary computing device 170 may additionally be a secondary monitoring device 160.

The integration may additionally enable augmentation of the device interactions. Device control integration may include control over window or view positioning. For example, a window may be repositioned on a computer screen or a virtual object may be repositioned to promote better ergonomics and posture for the user secondary computing device. Another variation of device control integration can include messaging wherein notifications or alerts can be triggered on the secondary computing device 170. In one example, an application on a personal computing device can enable push notifications to be delivered to a personal computing device when poor ergonomic activity is detected during active use of the device as shown in FIG. 4.

In some variations, the secondary computing device 170 may include computing devices outside traditional computer-like devices such as vehicles, control panels (e.g., flight traffic control panels, industrial control panels, military control panels), medical devices making use of multiple machines. While the system may be used in reinforcing good ergonomics for these secondary monitoring devices 160 as well, the system may integrate or be used with an outside device to promote enhanced safety or operation of the device. For example, the system may be able to alert a user when their attention is diverted in an unsafe way while driving.

In some variations, the application 150, the secondary monitoring device 160, and/or the secondary computing device 170 may be integrated as one or any suitable number of computing devices or systems. In a single device variation, the user interface of the application 150 may be provided through the secondary computing device 170 where device usage is monitored, and the orientation of the secondary computing device 170 may be monitored such that it also serves as a secondary monitoring device 160.

The biomechanical processing modules of the system function to characterize user motion and biomechanical state. The biomechanical signals can be used for monitoring activity or as part of a device input. A biomechanical processing module is preferably configured to process and transform sensed kinematic data into: structured biomechanical user modeling, user state information, device state information, and/or other extracted data representations. In some variations, the biomechanical signals can be used in combination with detected device usage, speech detection, and/or other user inputs when interfacing with a computing device.

More generally, the biomechanical processing modules are kinematic processing modules, and, as such, some kinematic processing modules may track motion, orientation, and/or state of non-biomechanical properties. In particular, movement, orientation and state of the head-wearable item 120 can be characterized as a data signal by a kinematic processing module.

Various biomechanical/kinematic processing modules (i.e., processing modules) may be used in isolation or in combination. One variation of a biomechanical processing module generates a signal relating to biomechanical movement and state of a user. Other variations of biomechanical processing modules may provide generalized analysis such as a calorie burn estimation.

The processing modules may operate continuously. Alternatively, a subset of the processing modules may be selectively activated under different conditions. For example, locomotion-based biomechanical processing modules may be activated when a walking or running activity state is detected.

Modeling of the various biomechanical or kinematic signals can be used to make additional higher level assessments such as determining when a user has satisfied some condition of bad ergonomics (e.g., holding head in position for longer than some threshold of time).

A processing module of the set of processing modules can be applied to generating data signals used to track various activities such as: head orientation tracking, posture, locomotion, head gestures or action detection, activity classification and tracking, item-specific action detection, meta-metrics, and/or other forms of analysis.

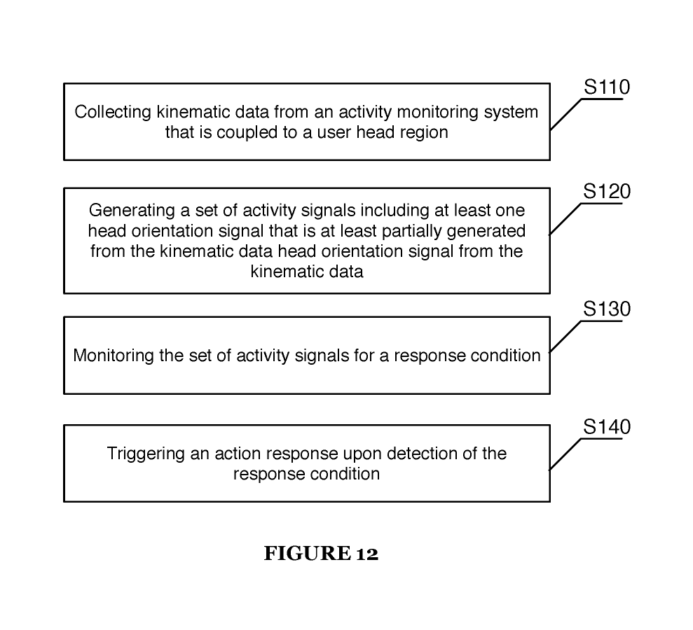

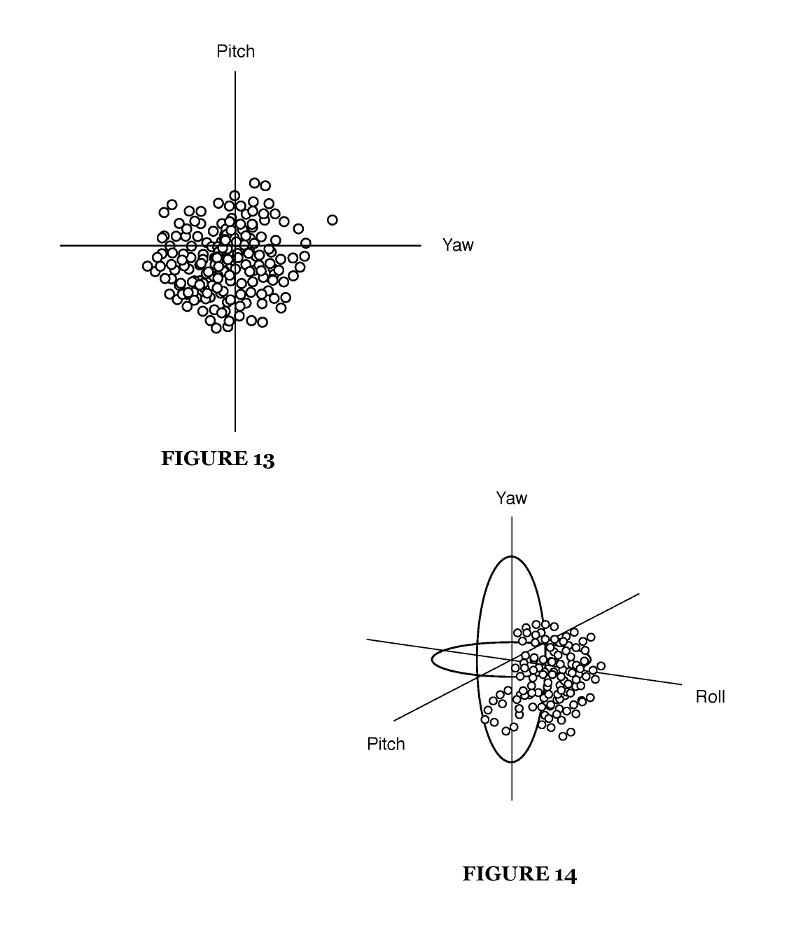

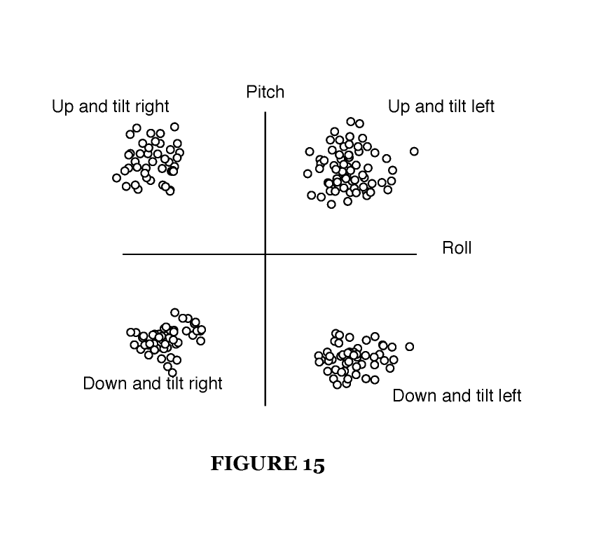

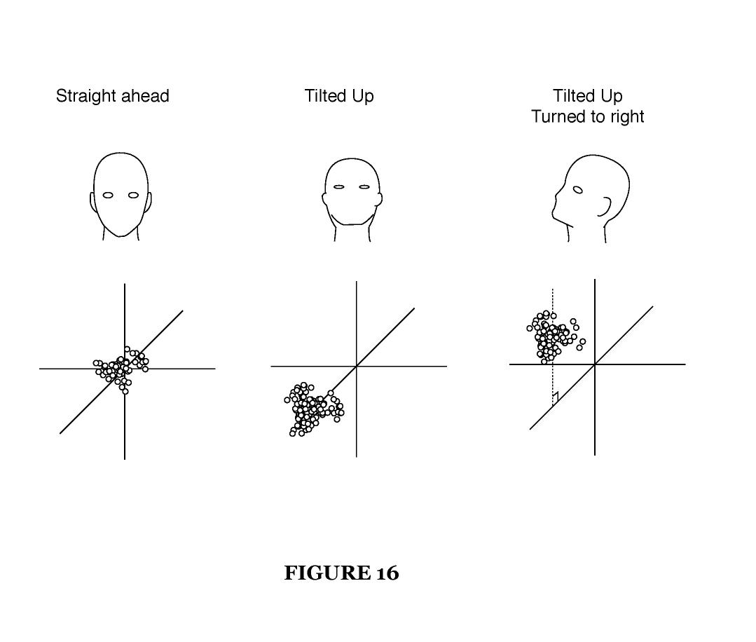

A head orientation tracking processing module can function to characterize the head orientation and/or motion. One head orientation tracking module can provide current head orientation data possibly represented by yaw, pitch, and roll rotation values. Additionally or alternatively, a head orientation tracking module can provide a historical orientation map that functions to characterize the range of motion and orientation patterns of the head/neck. This historical orientation map may be compiled from a collection of current head orientations over time. In one implementation, the orientation map can be an accumulative heat map data representation of head posture. In a basic version, the orientation map may be a measure of time at various orientation combinations. A two-dimensional version may have time accumulation over the last 24 hours for pitch/yaw as shown in FIG. 13. A three-dimensional example could have a counting metric (e.g., time, occurrence, or density distribution of occurrence) for combinations of pitch, roll, and yaw as shown in FIG. 14. Head orientation tracking may more generally track looking in general regions such as looking left, right, up, down, straight, etc. Generation of an orientation map can be used for detecting patterns of head postures overly used, underused, range of motion, and other patterns. For example, as shown in FIG. 15, different types of head positions may be labeled or classified through plotting on an orientation map. As shown in FIG. 16, through rotational dimensions of head orientation may result in tracking of head orientation to be mapped to different coordinates of an orientation map.

A posture processing module can function to characterize posture and more specifically neck/head posture. A posture processing module can be used to determine if a user is slouching from the neck and alert the user to adjust in an appropriate manner. A posture processing module of one variation can provide a measure of current posture's offset from a target posture. The target posture may vary between activities.

A locomotion processing module can function to characterize one or more properties of a user's walking, running, and/or other forms of striding or user movement. One locomotion processing module variation can detect and count steps. Steps can be counted by segmenting the kinematic data. Segmenting the kinematic data preferably classifies time windows of the data streams into consistently detected segments based on a repeated motion or predetermined motion. In the case of walking or running, steps can be identified and used to define the segments. Herein, step segments are used as the exemplary form of a segment, but a segment could alternatively be any portion of an action such as an arm stroke, a swing, a rowing motion, a pedal, or any suitable action of interest during an activity. Segmenting can use various techniques for step detection. In one variation, steps can be segmented and counted according to threshold or zero crossings of vertical velocity. A preferred approach, however, includes counting vertical velocity extrema.

Additionally, while walking or running, the eyewear can quantify additional dynamics such as: the amount of vertical oscillation, forward/backward braking forces, stride rate of a user, stride symmetry, and/or other stride-based biomechanical signals.

A head gestures or action detection processing module can function to detect particular actions. This may be used for detecting user input. For example, a processing module may be trained or otherwise configured for detection of nodding and/or shaking of the head. If integrated into a pair of smart glasses, a user can provide affirmative and negative directions by performing the appropriate gesture.

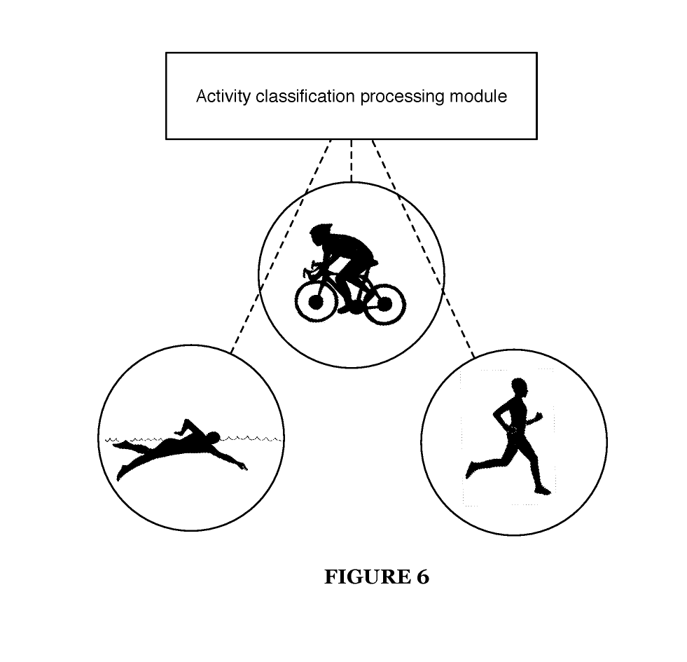

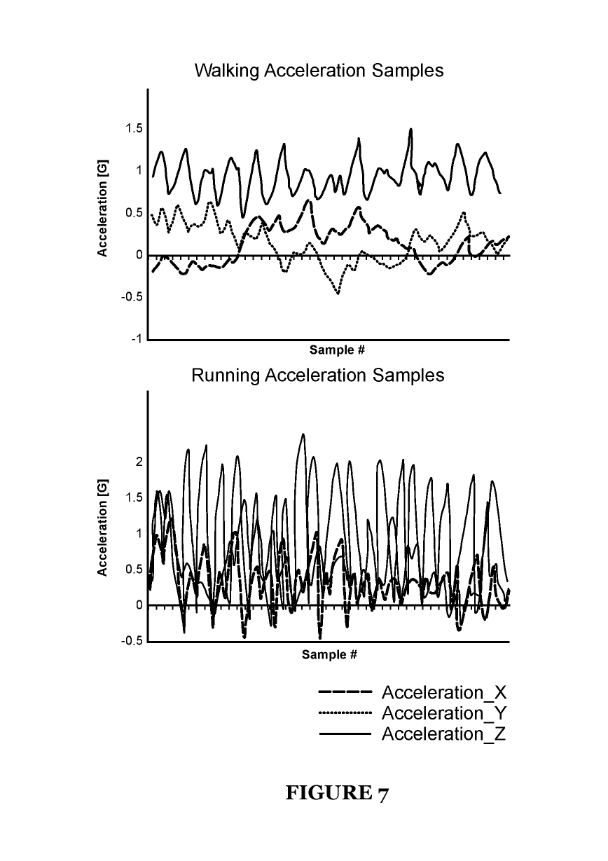

An activity classification or tracking processing module functions to predict current activity state from kinematic data. Various patterns in kinematic data over particular periods may be associated with different activities. Potential activities that can be classified and detected by an activity classification module can include standing, sitting, walking, running, driving, and the like as shown in FIG. 6. Such activities may additionally be further classified as different forms of activities. For example, an activity classification module can be used to classify sitting on a couch and sitting at a computer work station. As shown in FIG. 7, kinematic data may enable activity detection for activities such as walking and running. In one implementation, the two activities can be classified through the energy difference exhibited in the two activities.

Activity classification may be used in combination with other results of the processing modules. In one variation, the system can track where a user is looking and for how long during different activity states. This may be used to feed into how to deliver appropriate feedback. For example, classification of reading or being on a computer can trigger monitoring if the user's gaze has been fixed for too long. After certain conditions, such as remaining stagnant for longer than some threshold, haptic vibration or an alert can be sent to tell the user to stretch or rotate the neck to help prevent against neck pain.

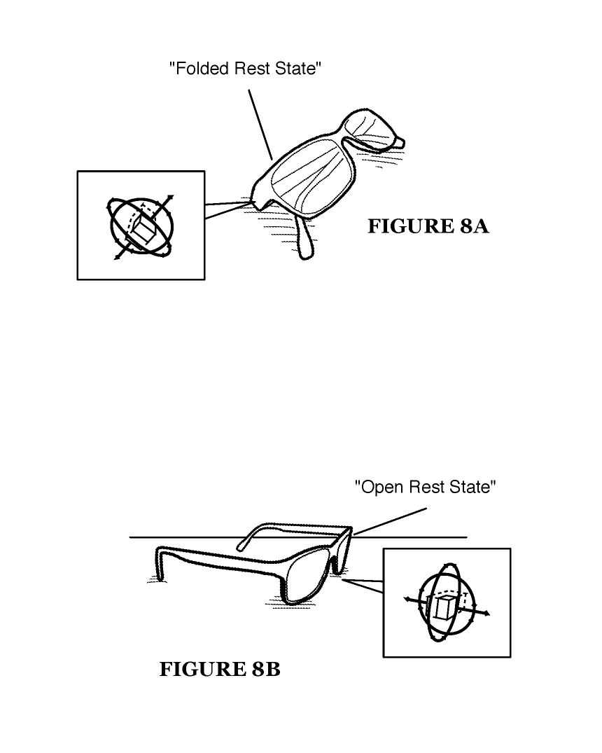

An item-specific action detection processing module functions to perform event detection that targets aspects of use for particular types of items. The item-specific action detection processing module is preferably based on the time of head-worn item 120. There may be different item-specific action detection processing modules for eyewear and headphones. The resulting analysis output can be used by device designers and providers to adjust product design for different use cases and demographics. This understanding could additionally be used in generating recommendations for current users or potential users. Glasses-specific action detection processing modules may include detecting adjustment of glasses, removing or putting on of glasses, setting glasses in a resting position (and optionally differentiating between setting on a flat surface, folding up and putting in a case, and the like), detecting mode of use (worn on top of head, tip of nose, etc.), and/or other item related events. In one example application, the resting state of glasses such as resting open on a flat surface or folded may be detectable states as shown in FIGS. 8A and 8B.

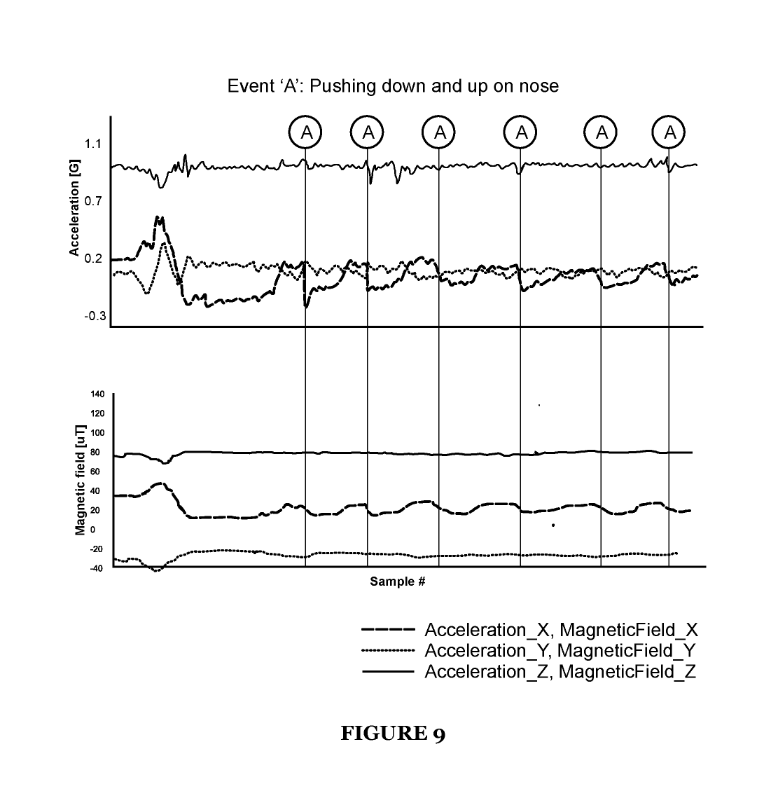

As shown in FIG. 9, even subtle item interactions may be detected and distinguished such as readjusting glasses to the bottom of the nose and readjusting to the top of the nose.

As shown in FIG. 10, kinematic data may exhibit patterns for subtle item interactions such as adjustments to glasses or nodding of a head up and down. For example, when nodding, the kinematic data may exhibit larger changes in acceleration in the X and Y axis compared to when the glasses are re-adjusted.

Meta-metric related processing modules functions to provide higher level analysis of other biometric or other forms of characterization of the kinematic data. The time spent walking, running, sitting and standing could be one form of analysis built on the detection of different activities. Another could be the conversion of activity to a calorie count during various activities.

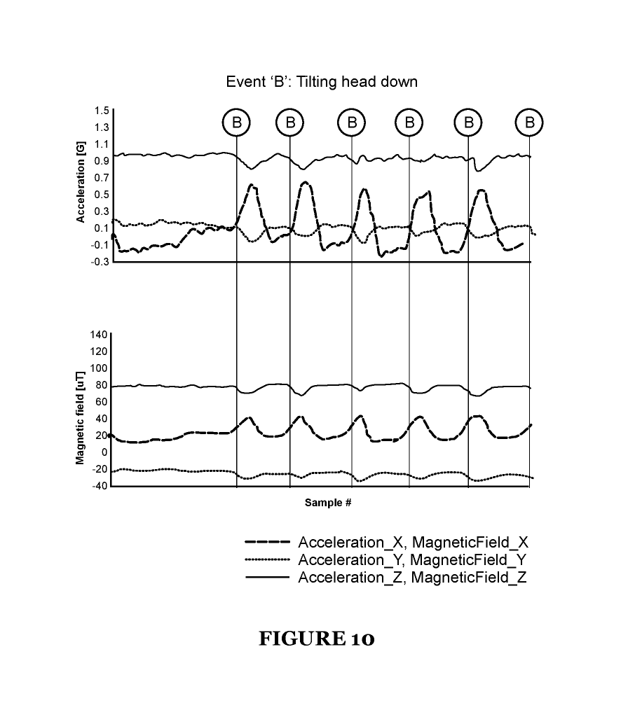

The various processing modules may be used in any suitable combination. As shown in FIG. 11, different kinematic data patterns of a user performing different actions such as looking around, walking, running, adjusting glasses can be detectable through a processing module.

### 3. Method

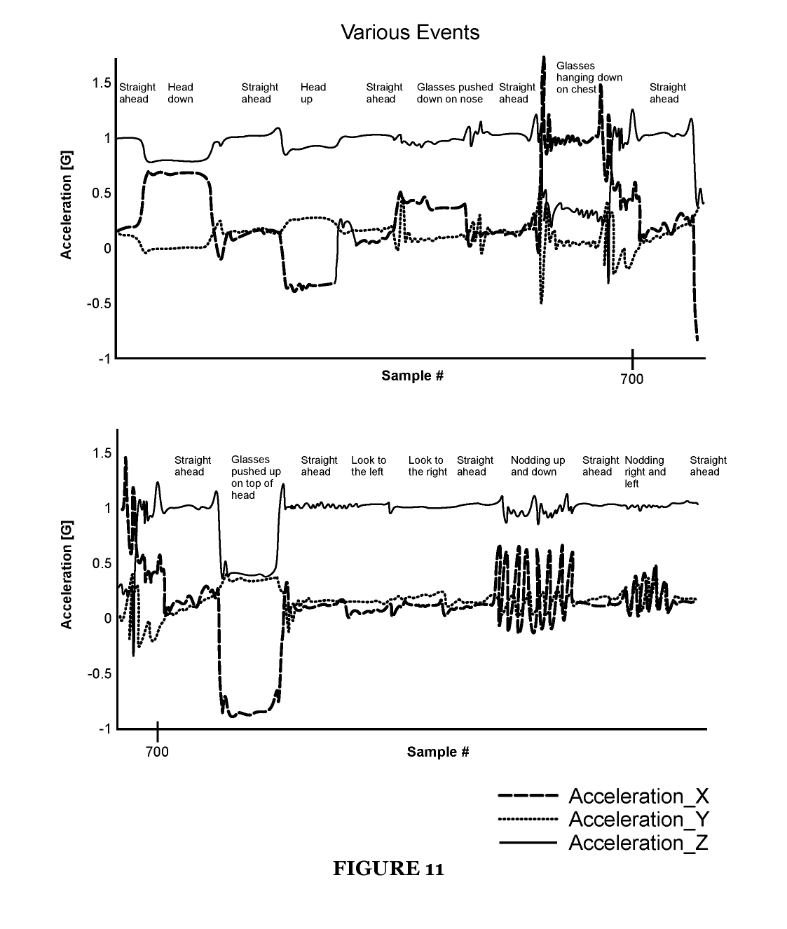

As shown in FIG. 12, a method for activity monitoring eyewear and head apparel of a preferred embodiment can include collecting kinematic data from an activity monitoring system that is coupled to a user head region S110; generating a set of activity signals including at least one head orientation signal that is at least partially generated from the kinematic data head orientation signal from the kinematic data S120; monitoring the set of activity signals for a response condition S130; and triggering an action response upon detection of the response condition S140.

The method is preferably directed at a head-worn device or system such as the one described above. However, any suitable system could alternatively implement the method. The method may be applied within a device to enable various features such as those related to activity analytics, product usage analytics, posture/ergonomic coaching, augmentation of a second computing device, and/or other applications.

Block S110, which includes collecting kinematic data from an activity monitoring system coupled to a user head region, functions to sense, detect, or otherwise obtain sensor data relating to motion and/or orientation of some portion of a user's head. The activity monitoring system preferably includes an activity monitoring system integrated with a head-wearable item as described above. In one variation, the activity monitoring system is integrated into eyewear. Eyewear may include vision corrective eyewear, sunglasses, safety goggles, smart glasses, a VR/AR headset, a glasses frame, or other forms of eyewear. In another variation, the activity monitoring system is integrated with audio headphones. The activity monitoring system may alternatively be integrated into any suitable type of item or product that may be coupled to the head of a user such as goggles, a VR/AR headset, a headband, a hairclip, a hat, a helmet, earrings, hearing aids, earbuds, a hood, or anything that can be worn on the head securely.

The kinematic data can be collected with an inertial measurement system that may include an accelerometer system, a gyroscope system, and/or a magnetometer. Preferably, the inertial measurement system includes a three-axis accelerometer and gyroscope. The kinematic data is preferably a stream of kinematic data collected over periods of time when a task is performed. The kinematic data may be collected continuously but may alternatively be selectively activated in response to different events. Some variations may include col

In one variation, data of the kinematic data is raw, unprocessed sensor data as detected from a sensor device. Raw sensor data can be collected directly from the sensing device, but the raw sensor data may alternatively be collected from an intermediary data source. In another variation, the data can be pre-processed. For example, data can be filtered, error corrected, or otherwise transformed. In one variation, in-hardware sensor fusion is performed by an on-device processor of the inertial measurement unit. The kinematic data is preferably calibrated to some reference orientation. In one variation, automatic calibration may be used as described in U.S. patent application Ser. No. 15/454,514 filed on 9 Mar. 2017, which is hereby incorporated in its entirety by this reference.

Any suitable pre-processing may additionally be applied to the data during the method. In one variation, collecting kinematic data can include calibrating orientation and normalizing the kinematic data.

An individual kinematic data stream preferably corresponds to distinct kinematic measurements along a defined axis. The kinematic measurements are preferably along a set of orthonormal axes (e.g., an x, y, z coordinate plane). In some variations, the axis of measurements may not be physically restrained to be aligned with a preferred or assumed coordinate system of the activity. Accordingly, the axis of measurement by one or more sensor(s) may be calibrated for analysis by calibrating the orientation of the kinematic data stream. One, two, or all three axes may share some or all features of the calibration, or be calibrated independently. Alternatively, the sensor(s) used in acquiring the kinematic data (e.g., an inertial measurement unit) may have substantially consistent orientation when worn by a user, in which case no orientation or alternative orientation approaches may be used. Eyewear in possibly other forms of head-wearable items may have substantially consistent orientation relative to the head. Accordingly, calibration may be performed during a device setup process and stored for long-term use. For example, a pair of smart glasses can have an initial calibration process executed when the user is wearing them during setup of the device.

The kinematic measurements can include acceleration, velocity, displacement, force, rotational acceleration, rotational displacement, tilt/angle, and/or any suitable metric corresponding to a kinematic property of an activity. Preferably, a sensing device provides acceleration as detected by an accelerometer and angular velocity as detected by a gyroscope along three orthonormal axes. The set of kinematic data streams preferably includes acceleration in any orthonormal set of axes in three-dimensional space, herein denoted as x, y, z axes, and angular velocity about the x, y, and z axes. Additionally, the sensing device may detect magnetic field through a three-axis magnetometer.

Calibrating the kinematic data can involve standardizing the kinematic data and calibrating the kinematic data to a reference orientation such as a coordinate system of the participant. The nature of calibration can be customized depending on the task and/or kinematic activity. The device including the sensor(s) can be attached or otherwise fixed into a certain position during an activity. That position can be static during the activity but may also be perturbed and change, wherein recalibration may be performed again.

Block S120, which includes generating a set of activity signals, functions to transform the kinematic data to conclusions on physical attributes relating to user or device activity.

In generating a set of activity signals there may be one generated activity signal or a plurality of activity signals. They may relate to each other or be distinct activity signals. Generating a set of activity signals may include tracking head orientation, measuring posture, generating a set of locomotion biomechanical signals, classifying activity state, detecting item-specific activity, and/or other forms of detection. The set of activity signals may be applied in various ways during monitoring and the triggering of actions.

Tracking head orientation functions to generate a measurement or set of measurements that characterize the orientation and/or position of the head. This can preferably be used to characterize at least one of side-to-side tilt (i.e., head roll), side-to-side rotation (i.e., head yaw), and/or up-down angle (i.e., head pitch) as shown in FIG. 3. As described, the kinematic data may be calibrated so that orientation of the sensor(s) can be mapped to head orientation. The head orientation can be recorded and tracked as a time series data set. The head orientation in some cases may be used in the calculation of other activity signals as is described below.

A single sensor may be able to provide one form of head orientation estimation. In some variations, tracking head orientation may include collecting base kinematic data from at least a second activity monitoring system coupled to a non-head region of the user as shown in FIG. 2, generating a base orientation from the base kinematic data, which functions to provide a frame of reference used in differentiating head motion and orientation from body and motion and orientation. In this variation, the head orientation can be generated relative to the base orientation. Base orientation changes will generally translate into similar orientation changes in the head when the head orientation is static relative to the body. Accordingly, the head orientation can be the offset between the base orientation and the tracked region orientation (i.e., measured head orientation prior to correction for body orientation). In particular, a base orientation and tracked region orientation can be generated, and the head orientation can be the difference between the base orientation and the tracked region orientation. The method may additionally include collecting kinematic data from a set of points on the body possibly beyond just a collection of base kinematic data. For example, one implementation may include sensing in the head region on the pelvis (or elsewhere on the trunk of the body) and/or on one or both feet/legs. The additional sensing points may be used in generating particular biomechanical signals.

The non-head region of the user preferably includes the trunk of the user (e.g., chest, stomach, back, pelvis, waist, etc.). The non-head region used for collection of base kinematic data may alternatively include the arms or legs. As the arms and legs may undergo more variations in motion and orientation, a base orientation may be inferred through the rest position or known positions of the arms and legs. The head-based activity monitoring system and the second activity monitoring system are preferably communicatively linked, wirelessly or through a wired connection. The second activity monitoring system may be an activity monitoring system substantially similar to the one used for monitoring of the head region, but is configured for attachment to a non-head region. Alternatively, the secondary activity monitoring system may be a different type of device such as a smart phone, smart watch, or any suitable computing device with at least one form of kinematic or orientation sensing capabilities. The type of kinematic data collected by the second activity monitoring system may be similar to the head-based activity monitoring system (e.g., both using three axis accelerometer and gyroscopic data). Alternatively, the second activity monitoring system may collect an alternative set of kinematic data. For example, the head-based activity monitoring system may collect three axis accelerometer and gyroscopic data while the base activity monitoring system could collect magnetometer data to determine global direction (and change in direction) of the body.

Measuring posture functions to generate a metric that reflects the nature of a user's posture and ergonomics.

In one variation, measuring posture can be an offset measurement of the head orientation relative to a target posture orientation. A target posture orientation may be pre-configured. For example, an activity monitoring system with a substantially consistent orientation when used by a user may have a preconfigured target posture orientation. Alternatively, a target posture orientation may be calibrated during use automatically. Target posture orientation may be calibrated automatically upon detecting a calibration state. A calibration state may be pre-trained kinematic data patterns that signal some understood orientation. For example, sitting down or standing up may act as a calibration state from which calibration can be performed. A target posture orientation may alternatively be manually set. For example, a user may position their head in a desired posture orientation and select an option to set the current orientation as a target orientation. Additionally, rotating the head in a circular motion, looking up, down or forward may act as a calibration state. In another variation, the target orientation may change depending on the current activity. Accordingly, measuring posture can include detecting a current activity through the kinematic data (or other sources), selecting a current target posture orientation for the current activity and measuring orientation relative to the current target posture orientation.

In one variation, measuring posture may include characterizing posture. Characterizing posture may not generate a distinct measurement, and instead classifies different kinematic states in terms of posture descriptors such as great posture, good posture, bad posture, and dangerous posture. Various heuristics and/or machine learning may be applied in defining classifications and detecting gesture classifications.

Another aspect of head posture may be sustained duration of any one orientation or posture state. Holding of a substantially stable posture can put excessive strain on the body. A static loading model may be used in measuring the quantity of static loading by detecting continuous holding of an orientation and/or the quantity of an orientation within a given window. For example, the amount of time a user has a “neck down” orientation over the course of a day may be accumulated and used as a measure of static loading for one group of orientations/posture states.

In addition, another posture model can track the amount of time the head is spent in various orientations such as looking forward, up, down, left, right and other various orientations. The feedback can be triggered based on the orientation such as the feedback may be generated after 30 seconds of looking in a downward direction, while looking to the left or right side may not generate a feedback signal, or may generate a feedback signal after a longer duration.

Another model may analyze the mobility of the head and neck region. Prompts for asking the user to rotate their head along some defined line, in an arc, in a circle; look side to side; or up and down can help the model assess the range of motion. Such checks can be made at various intervals to help measure the improvement or degradation of head and neck mobility. In addition, head/neck mobility can be assessed automatically by analyzing the various head motions and orientations made throughout the day.

Generating a set of locomotion biomechanical signals functions to transform one or more elements of the kinematic data into biomechanical characterizations of locomotion-associated actions. Here locomotion can include striding activities such as walking, jogging, or running. During other physical activities such as biking, rowing, swimming, and the like, the locomotion can include a metric relating to some aspect of the biomechanical performance of the task. In one variation, biomechanical signals may be generated in a manner substantially similar to that described in U.S. patent application Ser. No. 15/283,016, filed 30 Sep. 2016, which is hereby incorporated in its entirety by this reference.

Generating locomotion biomechanical measurements can be based on step-wise windows of the kinematic data—looking at single steps, consecutive steps, or a sequence of steps. In one variation, generating locomotion biomechanical measurements and more specifically gait biomechanical measurements can include generating a set of stride-based biomechanical signals comprising segmenting kinematic data by steps and for at least a subset of the stride-based biomechanical signals generating a biomechanical measurement based on step biomechanical properties. Segmenting can be performed for walking and/or running. In one variation steps can be segmented and counted according to threshold or zero crossings of vertical velocity. A preferred approach, however, includes counting vertical velocity extrema. Another preferred approach includes counting extrema exceeding a minimum amplitude requirement in the filtered, three-dimensional acceleration magnitude as measured by the sensor.

The set of stride-based biomechanical signals can include cadence, ground contact time, braking, forward oscillation, upper body trunk lean, step duration, stride or step length, step impact or shock, body loading ratio, step and/or stride length, swing time, double-stance time, activity transition time, stride symmetry, left and right step detection, pelvic dynamics (e.g., lateral oscillation, vertical oscillation, rotations, etc.), motion paths, and/or other features. Other health related biomechanical measurements can relate to balance, turn speed, tremor quantification, shuffle detection, variability or consistency of a biomechanical property, and/or other suitable health related biomechanical properties.

Cadence can be characterized as the step rate of the participant.

Ground contact time is a measure of how long a foot is in contact with the ground during a step. The ground contact time can be a time duration, a percent or ratio of ground contact compared to the step duration, a comparison of right and left ground contact time or any suitable characterization.

Braking or the intra-step change in forward velocity is the change in the deceleration in the direction of motion that occurs on ground contact. In one variation, braking is characterized as the difference between the minimum velocity and maximum velocity within a step, or the difference between the minimum velocity and the average velocity within a step. Braking can alternatively be characterized as the difference between the minimal velocity point and the average difference between the maximum and minimum velocity. A step impact signal may be a characterization of the timing and/or properties relating to the dynamics of a foot contacting the ground.

Upper body trunk lean is a characterization of the amount a user leans forward, backward, left or right when walking or running.

Step duration is the amount of time to take one step. Stride duration could similarly be used, wherein a stride includes two consecutive steps.

Step length is the forward displacement of each foot. Stride length is the forward displacement of two consecutive steps of the right and left foot.

Swing time is the amount of time each foot is in the air. Ground contact time is the amount of time the foot is in contact with the ground.

Double-stance time is the amount of time both feet are simultaneously on the ground during a walking gait cycle.

Activity transition time preferably characterizes the time between different activities such as lying down, sitting, standing, walking, and the like. A sit-to-stand transition is the amount of time it takes to transition from a sitting state to a standing state.

Stride symmetry can be a measure of imbalances between different steps. It can account for various factors such as stride length, step duration, pelvic rotation, and/or other factors. In one implementation, it can be characterized as a ratio or side bias where zero may represent balanced symmetry and a negative value or a positive value may represent left and right biases respectively. Symmetry could additionally be measured for different activities such as posture symmetry (degree of leaning to one or another side) when standing.

Left and right step detection can function to detect individual steps. Any of the biomechanical measurements could additionally be characterized for left and right sides.

Pelvic dynamics can be represented in several different biomechanical signals including pelvic rotation, pelvic tilt, and pelvic drop. Pelvic rotation (i.e., yaw) can characterize the rotation in the transverse plane (i.e., rotation about a vertical axis). Pelvic tilt (i.e., pitch) can be characterized as rotation in the sagittal plane (i.e., rotation about a lateral axis). Pelvic drop (i.e., roll) can be characterized as rotation in the coronal plane (i.e., rotation about the forward-backward axis).

Upper body trunk lean is a characterization of the amount a user leans forward, backward, left or right when walking.

Vertical oscillation of the pelvis is characterization of the up and down bounce during a step (e.g., the bounce of a step).

Lateral oscillation of the pelvis is the characterization of the side-to-side displacement during a stride possibly represented as a lateral displacement.

The motion path can be a position over time map for at least one point. Participants will generally have movement patterns that are unique and generally consistent between activities with similar conditions.

Balance can be a measure of posture or motion stability when walking, running, standing, carrying, or performing any suitable activity.

Turn speed can characterize properties relating to turns by a user. In one variation, turn speed can be the amount of time to turn. Additionally or alternatively turn speed can be characterized by peak velocity of turn, and/or average velocity of turn when a user makes a turn in their gait cycle.

Biomechanics variability or consistency can characterize variability or consistency of a biomechanical property such as of the biomechanical measurements discussed herein. The cadence variability may be one exemplary type of biomechanical variability signal, but any suitable biomechanical property could be analyzed from a variability perspective. Cadence variability may represent some measure of the amount of variation in the steps of the wearer. In one example, the cadence variability is represented as a range of cadences. The cadence variability may be used for particular activities such as running or walking.

A subset of the biomechanical signals may rely on secondary monitoring systems coupled to different locations in acquiring the various kinematic data streams.

In some cases, the collection and calculation of biomechanical signals may be used for general feedback outside of head-related information. For example, sensing and detecting vertical oscillation from a head-based activity monitoring system can be used for providing running form feedback.

The above biomechanical measurements can have particular applicability to walking, running, and standing use-cases. Alternative use cases may use alternative biomechanical measurements relating to acceleration, deceleration, change of direction, jump duration, and other suitable properties of performing some activity.

Detecting a current physical activity state functions to classify a current or previous activity of a user. Detecting a current physical activity state preferably includes analyzing kinematic data and detecting physical activity state from patterns in the kinematic data. The current and/or physical patterns of the physical activity state can be monitored as part of a response condition and used in triggering different response actions. Examples of detectable physical activity states can include driving, standing, sitting (e.g., sitting in a couch, sitting at a desk, and the like), striding (e.g., walking, running, jogging, and the like), lying down, and the like. In one variation, each activity state classification within a set of activity state classifications can be configured with different head orientation conditions that are used with the response condition. In this way, different head orientations (and the activity data in general) can be monitored differently depending on the current activity. These variations preferably involve detecting an activity state and selecting at least one response condition that is associated with the detected activity. Then in block S130, the set of activity signals is monitored based on the selected response condition. For example, detecting sitting at a desk may initiate monitoring for desk-specific response conditions. In another example, when the current activity state classification is driving, a driving-specific set of response conditions can be selected for monitoring. The driving response conditions may promote awareness of surroundings, avoiding distractions like looking at a phone or the radio, and/or avoiding drowsiness. In another example, when the current activity state classification is running, a running-specific set of response conditions can be selected for monitoring. The running response conditions may promote a base posture of the head looking out in front of the runner instead of down at the ground. This could be used to trigger feedback when a runner has been running with his or her head down for a long duration. Various other running response conditions may also be monitored such as tracking various biomechanical signals and comparing them to different targeted goals. In some variations, various biomechanical signals may be monitored in combination with head orientation. In one variation, running specific biomechanical signals such as cadence and vertical oscillation can be tracked and coached in combination with monitoring head orientation. For example, a runner could receive feedback to target a particular cadence and/or vertical oscillation metric but while maintaining head orientation in a posture where the runner is looking up at the horizon. In another variation, pelvic, core or foot orientation and biomechanical signals measured by a secondary monitoring device may be tracked in combination with head orientation. Posture feedback could be based on the combined postures of the back, pelvis and the head.

Detecting an item-specific action may act similarly to biometric signal sensing but instead is targeted at aspects of the item to which the activity monitoring system is integrated such as glasses, headphones, and the like. For glasses, detecting an item-specific action may include detecting putting on of glasses, removing of glasses, resting of glasses with frames open, resting of glasses with frames closed, placement of glasses in a case, correcting of position of glasses on the nose, and/or other forms of glasses use. In the case of resting position and the state of resting (frames open, closed, in the case, etc.), the state may be detected by associating a particular orientation to those states and detecting those orientations. For example, glasses resting on a flat surface with the frames closed will have a particular orientation that is generally unique to other uses as shown in FIGS. 8A and 8B. When that orientation is seen for an amount of time satisfying a duration threshold, then the closed frame resting position can be detected.

Generating an activity signal may additionally include other forms of detection and signal generation such as detecting gestures or forming meta-metrics from sensed information.

In one variation, the method may include detecting head and/or action gestures. Gestures such as nodding, shaking, tilting, and the like may be used for input to this or other systems. In one variation, a detected gesture can be used to acknowledge or select a response to an action response in S140. For example, haptic feedback (or some form of feedback) may be triggered to signal to the user to use better posture. Detecting a head gesture within a window of time, can be used in selecting a feedback option such as signaling to the system “feedback acknowledged”, “incorrect feedback”, “sleep the feedback” (i.e., don't provide this type of feedback for some preset amount of time), “recalibrate feedback”, and the like.

In one variation, various initially calculated activity signals may be processed and analyzed in generating meta-metrics. For example, burnt calories may be calculated from various biometric signals.

Block S130, which includes monitoring the set of activity signals for a response condition, functions to detect patterns in the activity signals that can be used in altering some system in block S140. The set of activity signals may include any suitable subset of activity signals such as those described above or any other suitable activity signals.

Posture related activity signals may be monitored with response conditions configured to promote good or improved posture. In one variation, monitoring the set of activity signals can include detecting head orientations satisfying an undesired head posture condition. The head posture condition can be orientation, sustained duration of orientation (e.g., holding a head position in an orientation range for some duration of time), intermittent duration orientation (e.g., how long head was in a position over the course of 10 minutes, hour, day, etc.), and the like. Feedback activation or any suitable action can be triggered in response to satisfying the undesired head posture condition. In one variation, monitoring the set of activity signals can include monitoring orientation coverage of head orientation within a graded range of an orientation map and identifying posture biases. This can function to enable the method to trigger action responses in response to range of motion and orientation tendencies. Posture biases can include orientations/postures that are more dominant for the user (e.g., main head orientations) and/or those that are less dominant (e.g., lesser used orientations and/or never used orientations). As discussed, an orientation map can be a multi-dimensional representation (e.g., a heat map) of head posture patterns across different head orientation positions. Various response conditions could be based on coverage exhibited in the orientation map. A healthy or target orientation map may have some defined patterns. In some variations, the orientation map will preferably have some three dimensional form with central common head orientations in the middle of that having higher use and those on the edge of normal flexibility having lower use but some level of usage. A healthy orientation map may additionally be characterized by uniformity without high contrast within the orientation map indicating an unusual concentration of the head being a particular orientation region. A healthy orientation map can additionally be analyzed for symmetry. A healthy individual will generally have similar flexibility in opposite sides, which may be reflected in the left-right symmetry of the orientation map. If the magnitude of a head orientation metric (e.g., duration, density distribution or occurrence count) in a particular region of the orientation map exceeds a threshold then the user may be overly using that head posture and can be delivered feedback to move the head in other orientations. If the magnitude of a head orientation metric (e.g., duration, density distribution or occurrence count) in a particular region of the orientation map is below a threshold then the user may be directed to move their head into that orientation and/or performing exercises/stretches to expand flexibility to be able to move their head to that orientation. The orientation map may additionally be used to generate recommendations such as generating an object rearrangement. For example, when in a desk or computer using activity, the orientation map may be used to detect unhealthy object usage and can generate a recommendation to reposition computer monitors or other pieces of equipment.

In some variations, the orientation map can additionally provide a useful posture visualization artifact and can be presented as an additional or alternative output of the method. An orientation map summarizing hourly, daily, past 24-hours, monthly, all time, and/or any time frame of data may be generated and presented in a companion application. Orientation maps could additionally be grouped by detected activities. In another variation, a time series orientation map that represents orientation map states at different points in time can additionally be presented. This could be an animation or a navigable visualization. For example, a user could view an animation showing their head orientation map as a function of time and/or select a particular time of day to see the orientation map at that time. The method may also include annotating the orientation map representation with activity, geo-location, and/or other forms of contextual meta data such as calendar events.

Response conditions are preferably configured for different patterns in the orientation coverage. The orientation map is preferably graded or weighted to account for the fact that some orientations or generally more commonly used than others. Alerts, coaching, and/or directed exercises may be triggered in block S140 to counteract the posture biases. For example, a user that rarely looks to their sides may be coached to look left and right periodically. Similarly, a user that has their head tilted at an orientation weighted as an infrequent position, but has their head in that position for a large amount of time in a time window may be alerted to use caution in their posture. Similarly, the response conditions may be based on the level of static loading over a defined time window. The level of static loading is preferably based on the temporal pattern of the activity signals and in particular the head orientation. Long sustained head orientations may result in stress and fatigue especially when the head orientation is one of poor or non-ideal posture. There may be a static loading model that accounts for breaks in head orientations and variety of head orientations that may serve to relieve stress and fatigue of different parts of the body (e.g., muscles and the like).

In addition, configurations can be set by the user to focus on improving a particular posture orientation or decreasing the amount of time in a particular orientation. For example, the user may want to reduce the amount of time looking down at their computer. Computer gaze detection, phone gaze detection, and/or other defined posture models can be monitored for particular patterns of posture orientation. The Posture orientation models may define particular posture orientations The user may also configure the duration of time a user can be allowed to look at a specific orientation before receiving a feedback response. Additionally, a user can configure goals to try to achieve the amount of time in a particular orientation, range of motion, or overall mobility.

Biomechanical signals may have response conditions based on various patterns or targets for biomechanical signals.

As described above, the response conditions may change based on the detected activity classification.

Item-specific activity may have associated response conditions. Item-specific activity may be used for collection of data for analytics. Accordingly, the occurrence of an item-specific activity may be the basic condition used to trigger some response action.

In one variation, the method may be used in connection with a second computing device to augment interactions with that second computing device. Such a variation may include monitoring user device interaction at the second computing device wherein the response condition is at least partially based on the user device interaction. The second computing device may be a distinct physical device such as a smart phone or computer. The second computing device may alternatively be a computing system integrated with the head-wearable item but providing a second objective. For example, the second computing device could be a VR/AR computing system integrated in the same head-wearable item. Activity with the VR/AR environment can be monitored and/or augmented based in part on the head orientation. In particular, the head orientation and the user device interaction state are the inputs of the response condition. This variation may be used to know that active use of a device is accompanied by particular head orientation, which may be used to detect bad device usage and/or to reward good device usage. For example, a response condition can be based on detected active use of the secondary computing device while head orientation satisfies a downward orientation condition (e.g., has a downward angle). This may be used to detecting when people are using the poor posture associated with use of smart phones and tablets. Monitoring user device interaction may be a simple detection of active use (e.g., the screen is on, user input is periodically happening, etc.) Monitoring user device interaction may additionally include detecting orientation of the secondary computing device (e.g., reading kinematic data from an inertial measurement unit of the computing device. The action response associated with this response condition can be some form of feedback delivered to the user. Alternatively, the action response can include feedback to the secondary computing device. For example, triggering the action response may include initializing a notification communicated to the secondary computing device. In another variation, the triggering of an action response in block S140 may include augmenting operation within the secondary computing device. In one example, this can include moving of user interface elements as shown in FIG. 5. The user interface elements are preferably moved to alter the position of a user's focus so as to counteract poor posture. For example, on a desktop computer or a virtual reality user interface, a user interface element (such as text box or window) can be moved from a low position to a high position so that the user can use better posture.

Block S140, which includes triggering an action response upon detection of the response condition, functions to perform an action based on the activity signals. The various activity signals that can be detected in association with the head-worn item may enable a variety of forms of interactive applications such as triggering feedback, guiding exercises, augmenting secondary computing devices, and the like.

In one variation, triggering the action response can include triggering a feedback alert. The feedback alert can be haptic feedback, an audio alert, a visual alert, and/or any suitable type of alert. The type and variety of feedback alerts are preferably mapped to different forms of feedback. For example, there can be one form of feedback alert for good posture and a second form for bad posture.

In another variation, triggering the action response can include directing an exercise interaction. Exercise interactions can be used as a form of feedback alert as above. The exercise interaction is preferably selected to counteract some issue detected in the response condition. For example, different forms of bad posture may have different exercises triggered to stretch or strengthen different parts of the body.

In one variation in association with the response condition, a set of exercises is generated in response to the monitored activity signals. The set of exercises are preferably customized to address the particular head orientation and activity patterns observed in the kinematic data. Triggering an action response can include communicating or directing the set of exercises with coordinated timing. The set of exercises are preferably communicated over spaced intervals as discussed above. As neck exercises can be brief exercises, the various exercises can be dispersed across the course of a day without significantly interfering with normal activity. Additionally, directing the set of exercises can comprise adjusting the order, duration, repetitions, and/or other aspects when communicating the exercises.

Delivery of exercise interactions as well as feedback alerts may be timed to enhance user susceptibility. This can be based on heuristics, a machine learning model, or any suitable approach. For example, exercise interactions may be delivered not during the poor posture but during a particular activity like walking. A user may be less distracted at those periods and more open to performing the activity.

As discussed above, triggering an action response may also be used in augmenting operation of a secondary computing device. A notification, message or instruction can be communicated to the second computing device that then preferably acts on that communication.

In some cases, where the head-worn item is a computing device, such as a smart glasses or a VR/AR headset, the operation of that computing device may be augmented in a substantially similar manner. As discussed above, augmenting a computing device may include altering of one or more user interface elements. This could similarly be done for the item-worn computing device. For example, operation of the VR/AR headset (or the driving computing unit) can be updated based on the action response so that the visual organization of the displays is reoriented to visually counter balance head orientation. If a UI widget is placed low in the field of view, then that UI widget may be moved higher so that a user does not need to look down.

In some cases, the action response may be the collection and organization of analytics related to head orientation and the activity signals. The activity signals may be communicated and accessed through an internet hosted data platform.

The systems and methods of the embodiments can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor but any suitable dedicated hardware device can (alternatively or additionally) execute the instructions.

As a person skilled in the art will recognize from the previous detailed description and from the figures and claims, modifications and changes can be made to the embodiments of the invention without departing from the scope of this invention as defined in the following claims.

We claim:

1. A method comprising:
collecting kinematic data from an activity monitoring system that is coupled to a user head region
generating a set of activity signals that comprises at least one head orientation signal that is at least partially generated from the kinematic data head orientation signal from the kinematic data;
monitoring the set of activity signals for a response condition; and
triggering an action response upon detection of the response condition.

2. The method of claim 1, wherein the activity monitoring system is integrated into eyewear.

3. The method of claim 1, wherein the activity monitoring system is integrated with audio headphones.

4. The method of claim 1, further comprising collecting base kinematic data from a second activity monitoring system coupled to a non-head region of the user; generating a base orientation from the base kinematic data from the second activity monitoring system; and wherein the head orientation is generated as an orientation metric relative to the base orientation.

5. The method of claim 1, wherein monitoring the set of activity signals for a response condition comprises monitoring orientation coverage of the head orientation within a graded range of motion map; and wherein the action response is feedback on head range of motion.

6. The method of claim 1, wherein generating a set of activity signals further comprises analyzing kinematic data and detecting a current physical activity state; and wherein monitoring the set of activity signals for a response condition is conditional in part on the current physical activity state.

7. The method of claim 6, wherein the physical activity state is one of a set of physical activity state classifications comprising: driving, standing, sitting, and striding.

8. The method of claim 6, wherein each activity state classification has at least one configured head orientation condition; and wherein monitoring the set of activity signals for a response condition is at least partially based on head orientation conditions selected based on the current activity state classification.

9. The method of claim 8, wherein, during a current activity state classification of driving, monitoring the set of activity signals for a response condition comprises selecting a driving set of response conditions for active monitoring.

10. The method of claim 8, wherein during a current activity state classification of running, monitoring the set of activity signals for a response condition comprises selecting a running set of response conditions for active monitoring.

11. The method of claim 1, further comprising monitoring user device interaction at a second computing device; and wherein the response condition is at least partially based on the user device interaction and the head orientation.

12. The method of claim ii, wherein monitoring user device interaction comprises detecting active use of the second computing device; and wherein the response condition is further conditional on detection of active use while head orientation satisfies a downward orientation.

13. The method of claim 11, wherein triggering the action response comprises initializing a notification communicated to the secondary computing device.

14. The method of claim 11, wherein triggering the action response comprises moving a user interface element of the secondary computing device.

15. The method of claim 1, wherein the response condition is based in part on a level of static loading as defined by the temporal pattern of the activity signals.

16. The method of claim 1, wherein triggering the action response comprises triggering a feedback alert.

17. The method of claim 16, wherein monitoring the set of activity signals for a response condition can include detecting head orientation satisfying an undesired head posture condition; wherein the feedback is triggered in response to satisfying the undesired head posture condition.

18. The method of claim 1, wherein triggering the action response comprises directing an exercise interaction.

19. The method of claim 18, further comprising generating a set of exercises in response to the monitored activity signals; and wherein triggering an action response comprises directing the set of exercises with coordinated timing.

20. The method of claim 19, wherein directing the set of exercises comprises communicating the exercises over spaced intervals.
